SEQUENCE LISTING (6 sequences)

SEQ ID NO: 1 - DNA, 30 bp, artificial sequence
  synthetic primer
aattaaacctgaagcttataaccatggagc

SEQ ID NO: 2 - DNA, 29 bp, artificial sequence
  synthetic primer
ggtgatcagcagacgtctgttgaaacatg

SEQ ID NO: 3 - DNA, 29 bp, artificial sequence
  synthetic primer
gggatcaaaaacaagcttggggcaaatgc

SEQ ID NO: 4 - DNA, 29 bp, artificial sequence
  synthetic primer
aagatgtagtttgacgtcaactaagcatg

SEQ ID NO: 5 - DNA, 25 bp, artificial sequence
  synthetic primer
aattgaaccatctcgagtagcaccc

SEQ ID NO: 6 - DNA, 33 bp, artificial sequence
  synthetic primer
cccactccattccggactcgggattccacctga

(19) United States
(12) Patent Application Publication
(10) Pub. No.: US 2004/0033604 A1
(43) Pub. Date: Feb. 19, 2004

(54) Recombinant lentiviral vectors pseudotyped in envelopes containing filovirus binding domains

(76) Inventors: Gary Kobinger, Philadelphia, PA (US); James M. Wilson, Gladwyne, PA (US)

Correspondence Address: HOWSON AND HOWSON, ONE SPRING HOUSE CORPORATION CENTER, BOX 457, 321 NORRISTOWN ROAD, SPRING HOUSE, PA, 19477, US

(21) Appl. No.: 10/257,960

(22) PCT Filed: Apr. 20, 2001
(86) PCT No.: PCT/US01/12880

(51) Int. Cl. C12N 15/867; C12N 7/00; A61K 48/00

(52) U.S. Cl. 435/456; 435/235.1; 424/93.2

(57) ABSTRACT

Recombinant transfer viruses, comprising an HIV minigene carrying a desired molecule, packaged in an envelope containing at least the binding domain of the ebola envelope protein, are described. Also described are methods of producing these transfer viruses and methods of using these viruses to deliver genes to selected target cells. These transfer viruses are particularly useful for delivery of molecules, in vivo, to lung cells following intracheal delivery or for delivery of molecules, ex vivo, to macrophages and dendritic cells.

This work was funded, in part, by grants from the NIH [NIDDK P30 DK47757-08 and NIAMS P01 ARJNS43648-05]. The US government has certain rights in this invention.

### BACKGROUND OF THE INVENTION

The invention relates generally to recombinant viruses useful for delivery of transgenes to selected host cells and to methods of producing same.

Retrovirus vectors derived from oncoretroviruses such as murine leukemia virus (MLV) have been used for gene therapy applications. However, a significant problem with such retrovirus vectors is their requirement for proliferating (i.e., dividing) target cells for integration. HIV and other lentiviruses have been described as being useful for gene delivery into non-dividing cells (J. Naldini et al. Science, 272:263-267 (Apr. 12, 1996)). However, there are significant safety concerns associated with use of many of the described HIV vectors, because recombination between the envelope packaging construct and transfer vector can generate replication competent HIV that may disseminate throughout the host.

In order to address this and other safety concerns HIV vectors have been packaged in the glycoprotein of a vesicular stomatitis virus (VSVG). Unfortunately, this construct has limited usefulness for delivery of genes into the lungs of the host, as the construct can transduce polarized airway epithelial cells only from the basolateral side, thus requiring invasive procedures for its use as a delivery vehicle.

What is needed is a safe vector useful for delivery of a therapeutic gene to a selected target cell. Further, what is desirable is a vector which can readily transduce the target cell using procedures in which invasiveness is minimized.

### SUMMARY OF THE INVENTION

The present invention provides a recombinant transfer virus, in which a lentiviral minigene is packaged in a heterologous envelope comprising the binding domain of a filovirus envelope protein. The envelope may be a full-length filovirus envelope protein or a fusion protein comprising the binding domain of a filovirus envelope protein fused to a heterologous membrane domain of a viral envelope protein. Advantageously, the recombinant transfer virus of the invention minimizes the safety concerns that the lentivirus will from replication competent virus. Further, in certain embodiments, the recombinant transfer virus of the invention is particularly well adapted to delivery to mammalian lung cells, as the transfer virus infects from the apical side, permitting delivery via intracheal administration.

Thus, in one aspect, the invention provides a recombinant transfer virus useful for delivering a selected molecule to a host cell. This transfer virus contains a lentivirus minigene packaged in a heterologous envelope containing, at least, the packaging domain of a filovirus envelope protein. In one particularly desirable embodiment, the filovirus is ebola. The lentivirus minigene contains the lentivirus 5′ long terminal repeat (LTR) sequences, a molecule for delivery to a host cell, and a functional portion of the lentivirus 3′ LTR sequences. In one embodiment, the minigene further contains functional lentiviral RRE sequences.

In another aspect, the invention provides a host cell containing the recombinant transfer virus of the invention.

In still another aspect, the invention provides methods of producing the recombinant transfer virus in vitro, or using a packaging cell. In one embodiment, the recombinant transfer virus is cultured in a packaging cell lentivirus packaging sequences, a nucleic acid molecule encoding an envelope protein containing a filovirus envelope binding domain under the control of regulatory sequences which direct expression of the envelope protein in the host cell, and a lentivirus minigene as described above. The lentivirus packaging sequences include a psi packaging signal, lentivirus gag sequences, lentivirus pol sequences, and lack the ability to express functional lentivirus envelope proteins. The host cell is cultured under conditions which permit packaging of the lentivirus minigene carrying the molecule in the envelope protein.

In yet another aspect, the invention provides a packaging cell containing the lentivirus packaging sequences, the lentiviral minigene and the nucleic acid molecule encoding the envelope protein.

In still another aspect, the invention provides a method of treating a patient with a selected transgene or other molecule, where the method involves transducing the cells of the patient with the recombinant virus of the invention. This method may be performed in in vivo or ex vivo.

In yet a further aspect, the invention provides a method of delivering a transgene or other molecule to the apical cells of the lung, in which the method involves administering a recombinant virus of the invention intratracheally.

### BRIEF DESCRIPTION OF THE DRAWINGS

FIG. 1 is a bar chart illustrating green fluorescent protein (GFIP) expression by a panel of pseudotyped lentiviruses 4 days after apical or basolateral application of 50 μl of partially concentrated viral stock.

FIG. 2 is a bar chart comparing gene transfer in the lungs of animals receiving vehicle or pseudotyped virus. Animals were sacrificed at the indicated timepoints and lungs were stained for β-gal expression. VSV-G-pseudotyped vector treated animals demonstrated minimal expression in the large airways at day 28 and 63. EboZ-pseudotyped vector treated animals demonstrated strong expression in the large airways and submucosal glands which decreased between day 28 and day 63.

### DETAILED DESCRIPTION OF THE INVENTION

The present invention provides a recombinant transfer virus, which avoids many of the problems of safety associated with lentiviral gene therapy vectors, and which is effective for transferring molecules to host cells including, notably, lung cells, dendritic cells, and macrophages, among others. Also provided are methods of producing these recombinant transfer viruses, as well as methods of using them for gene delivery in vitro and in vivo.

The inventors have found that recombinant transfer viruses of the invention, in particular viruses which contain an HIV vector pseudotyped in an ebola virus envelope protein, are able to efficiently transduce intact airway epithelia ex vivo and more importantly in vivo. These results indicate that recombinant transfer viruses of the invention can overcome physical and biochemical barriers present on normal airway epithelium. Furthermore, in a tracheal explant model of cystic fibrosis (CF), CF explanted airway could be efficiently transduced using the EboZ pseudotyped virus of the invention despite the presence of some mucus. An unexpected finding was that the EboZ-pseudotyped virus of the invention efficiently transduced submucosal glands in addition to the proximal and distal surface epithelia, and to a lower extent, alveolar epithelium. •-gal staining was observed in submucosal glands from transduced human airway explant as well as in C57B1/6 mice. Thus, the transfer viruses of the invention overcome barriers which are known to exist for gene transfer vectors of the prior art. Thus, the transfer viruses of the invention are particularly well suited for delivery of molecules to airway cells, e.g., for treatment of CF. Other advantages and uses of the transfer viruses of the invention are described below and will readily apparent to those of skill in the art.

### I. Recombinant Transfer Virus

Thus, in one embodiment, the invention provides a recombinant transfer virus composed of a lentivirus minigene packaged in a heterologous envelope comprising the binding domain of a filovirus envelope protein. The lentiviral minigene of the invention contains, at a minimum, lentivirus 5′ long terminal repeat (LTR) sequences, a molecule for delivery to the host cells, and a functional portion of the lentivirus 3′ LTR sequences. Optionally, the minigene may further contain a psi encapsidation sequence, RRE sequences or sequences which provide equivalent or similar function.

The heterologous molecule carried on the minigene for delivery to a host cell may be any desired substance including, without limitation, a polypeptide, protein, enzyme, carbohydrate, chemical moiety, or nucleic acid molecule which may include oligonucleotides, RNA, DNA, and/or RNA/DNA hybrids. In one embodiment, the heterologous molecule is a nucleic acid molecule which introduces specific genetic modifications into human chromosomes, e.g., for correction of mutated genes. In another desirable embodiment, the heterologous molecule comprises a transgene comprising a nucleic acid sequence encoding a desired protein, peptide, polypeptide, enzyme, or another product and regulatory sequences directing transcription and/or translation of the encoded product in a host cell, and which enable expression of the encoded product in the host cell. Suitable products and regulatory sequences are discussed in more detail below. However, the selection of the heterologous molecule carried on the minigene and delivered by the viruses of the invention is not a limitation of the present invention.

A. Lentiviral Elements

In selecting the lentiviral elements described herein for construction of the lentivirus minigene and the transfer virus of the invention, one may readily select sequences from any suitable lentivirus and any suitable lentivirus serotype or strain. Suitable lentiviruses include, for example, human immunodeficiency virus (HIV), simian immunodeficiency virus (SIV), caprine arthritis and encephalitis virus, equine infectious anemia virus, visna virus, and feline immunodeficiency virus (FIV). The examples provided herein illustrate the use of a minigene derived from HIV. However, FIV and other lentiviruses of non-human origin may also be particularly desirable. The sequences used in the constructs of the invention may be derived from academic, non-profit (e.g., the American Type Culture Collection, Manassas, Va.) or commercial sources of lentiviruses. Alternatively, the sequences may he produced recombinantly, using genetic engineering techniques, or synthesized using conventional techniques (e.g., G. Barony and R. B. Merrifield, THE PEPTIDES: ANALYSIS, SYNTHESIS & BIOLOGY, Academic Press, pp. 3-285 (1980)), with reference to published viral sequences, including sequences contained in publicly accessible electronic databases. In the following specification, it will be understood that a reference to lentiviral sequences involves any suitable means of obtaining the referenced sequences.

1. LTR sequences

The lentiviral minigene contains a sufficient amount of lentiviral long terminal repeat (LTR) sequences to permit reverse transcription of the genome, to generate cDNA, and to permit expression of the RNA sequences present in the lentiviral minigene. Suitably, these sequences include both the 5′ LTR sequences, which are located at the extreme 5′ end of the minigene and the 3′ LTR sequences, which are located at the extreme 3′ end of the minigene. These LTR sequences may be intact LTRs native to a selected lentivirus or a cross-reactive lentivirus, or more desirably, may be modified LTRs.

Various modifications to lentivirus LFRs have been described. One particularly desirable modification is a self-inactivating LTR, such as that described in H. Miyoshi et al, J. Virol., 72:8150-8157 (October 1998) for HIV. In these HIV LTRs, the U3 region of the 5′ LTR is replaced with a strong heterologous promoter (e.g., CMV) and a deletion of 133 bp is made in the U3 region of the 3′ LTR. Thus, upon reverse transcription, the deletion of the 3′ LTR is transferred to the 5′ LTR, resulting in transcriptional inactivation of the LTR. The complete nucleotide sequence of HIV is known, see, L. Ratner et al. Nature, 313(6000):277-284 (1985). Yet another suitable modification involves a complete deletion in the U3 region, so that the 5′ LTR contains only a strong heterologous promoter, the R region, and the U5 region; and the 3′ LTR contains only the R region, which includes a polyA. In yet another embodiment, both the U3 and U5 regions of the 5′ LTRs are deleted and the 3′ LTRs contain only the R region. These and other suitable modifications may be readily engineered by one of skill in the art, in HIV and/or in comparable regions of another selected lentivirus.

Optionally, the lentiviral minigene may contain a ψ (psi) packaging, signal sequence downstream of the 5′ lentivirus LTR sequences. Optionally, one or more splice donor sites may be located between the LTR sequences and immediately upstream of the ψ sequence. According to the present invention, the ψ sequences may be modified to remove the overlap with the gag sequences and to improve packaging. For example, a stop codon may be inserted upstream of the gag coding sequence. Other suitable modifications to the y sequences may be engineered by one of skill in the art. Such modifications are not a limitation of the present invention.

In one suitable embodiment, the lantiviral minigene contains lentiviral Rev responsive element (RRE) sequences located downstream of the LTR and ψ sequences. Suitably, the RRE sequences contain a minimum of about 275 to about 300 nt of the native lentiviral RRE sequences, and more preferably, at least about 400 to about 450 nt of the RRE sequences. Optionally, the RRE sequences may be substituted by another suitable element which assists in expression of gag/pol and its transportation to the cell nucleus. For example, other suitable sequences may include the CT element of the Manson-Pfizer virus, or the woodchuck hepatitis virus post-regulatory element (WPRE). Alternatively, the sequences encoding gag and gag/pol may be altered such that nuclear localization is modified without altering the amino acid sequences of the gag and gag/pol polypeptides. Suitable methods will be readily apparent to one of skill in the art.

2. Transgene

As stated above, in one desirable embodiment, the molecule carried by the lentiviral minigene is a transgene. The transgenic a nucleic acid molecule comprising a nucleic acid sequence, heterologous to the lentiviral sequences, which encodes a protein, peptide, polypeptide, enzyme, or another product of interest and regulatory sequences directing transcription and/or translation of the encoded product in a host cell, and which enable expression of the encoded product in the host cell. The composition of the transgene depends upon the intended use for the minigene and the pseudotyped virus of the invention.

For example, one type of transgene comprises a reporter or marker sequence which, upon expression, produces a detectable signal. Such reporter or marker sequences include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, membrane bound proteins including, for example, CD2, CD4, CD8, and the influenza hemagglutinin protein, as well as others well known in the art. Advantageously, high affinity antibodies to such proteins exist or can be made routinely, as can fusion proteins comprising a membrane bound protein appropriately fused to an antigen tag domain from, among others, hemagglutinin or Myc. These sequences, when associated with regulatory elements which drive their expression, provide signals detectable by conventional means. Such conventional means include enzymatic, radiographic, colorimetric, fluorescence or other spectrographic assays, fluorescent activated cell sorting assay and immunological assays, including ELISA, RIA and immunohistochemistry. For example, where the transgene comprises the LacZ gene, the presence of the transfer virus containing the lentiviral minigene is detected by assays for beta-galactosidase activity. Similarly, where the transgene is luciferase, transfer virus may be measured by light production in a luminometer.

However, desirably, the transgene contains a non-marker gene which can be delivered to a cell or an animal via the transfer virus of the invention. The transgene may be selected from a wide variety of gene products useful in biology and medicine, such as proteins, antisense nucleic acids (e.g., RNAs), or catalytic RNAs. The transfer viruses of the invention are useful for delivery of gene products and other molecules which induce an antibody and/or cell-mediated immune response, e.g., for vaccine purposes. Suitable gene products may be readily selected by one of skill in the art from among immunogenic proteins and polypeptides derived from viruses, as well as from prokaryotic and eukaryotic organisms, including unicellular and multicellular parasites. In another alternative, the recombinant transfer viruses of the invention are useful for delivery of a molecule desirable for study.

In one particularly desirable embodiment, the transfer viruses of the invention are useful for therapeutic purposes, including, without limitation, correcting or ameliorating gene deficiencies, wherein normal genes are expressed but at less than normal levels. The transfer viruses may also be used to correct or ameliorate genetic defects wherein a functional gene product is not expressed. A preferred type of transgene contains a sequence encoding a desired therapeutic product for expression in a host cell. These therapeutic nucleic acid sequences typically encode products which, upon expression, are able to correct or complement an inherited or non-inherited genetic defect, or treat an epigenetic disorder or disease.

Thus, the invention includes methods of producing a transfer virus which can be used to correct or ameliorate a gene defect caused by a multi-subunit protein. In certain situations, a different transgene may be used to encode each subunit of the protein. This is desirable when the size of the DNA encoding the protein subunit is large, e.g., for an immunoglobulin or the platelet-derived growth factor receptor. In order for the cell to produce the multi-subunit protein, a cell would be infected with transfer viruses containing each of the different subunits. Alternatively, different subunits of a protein may be encoded by the same transgenie. In this case, a single transgene would include the DNA encoding each of the subunits, with the DNA for each subunit separated by an internal ribosome entry site (IRES). This is desirable when the size of the DNA encoding each of the subunits is small, such that the total of the DNA encoding the subunits and the IRES is less than nine kilobases. Alternatively, other methods which do not require the use of an IRES may be used for co-expression of proteins. Such other methods may involve the use of a second internal promoter, an alternative splice signal, or a co- or post-translational proteolytic cleavage strategy, among others which are known to those of skill in the art.

Other useful gene products encoded by the transgene include hormones and growth and differentiation factors including, without limitation, insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factors (CTGF), basic fibroblast growth factor (bFGF), acidic fibroblast growth factor (aFGF), epidermal growth factor (EGF), transforming growth factor a (TGFα), platelet-derived growth factor (PDGF), insulin-like growth factors I and II (IGF-I and IGF-II), any one of the transforming growth factor β (TGFβ) superfamily comprising TGFβ, activins, inhibins, or any of the bone morphogenic proteins (BMP) BMPs 1-15, any one of the heregulin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Still other useful gene products include proteins that regulate the immune system including, without limitation, cytokines and lymiphokines such as thrombopoictin (TPO), interleukins (IL) IL-1α, IL-1β, IL-2, IL-3, IL-4, IL-5, IL-6, IL-7, IL-8, IL-9, IL-10, IL-11, IL-12, IL-13, IL-14, IL-15, IL-16, and IL-17, monocyte chemoattractant protein (MCP-1), leukemia inhibitory factor (LIF), granulocyte-macrophage colony stimulating factor (GM-CSF), Fas ligand, tumor necrosis factors α and β (TNFα and TNFβ), interferons (IFN) IFN-α, IFN-β and IFN-γ, stem cell factor, flk-2/flt3 ligand. Gene products produced by the immune system are also encompassed by this invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered MHC molecules including single chain MHC molecules. Useful gene products also include complement regulatory proteins such as membrane cofactor protein (MCP), decay accelerating factor (DAF). CR1, CR2 and CD59.

Yet other useful gene products include any one of the receptors for the hormones, growth factors, cytokines, lymphokines, regulatory proteins and immune system proteins. The invention encompasses receptors for cholesterol regulation, including the LDL receptor, HDL receptor, VLDL receptor, and the scavenger receptor. The invention also encompasses gene products such as the steroid hormone receptor superfamily including glucocorticoid receptors and estrogen receptors. Vitamin D receptors and other nuclear receptors. In addition, useful gene products include transcription factors such as jun, fos, max, mad, serum response factor (SRF). AP-1, AP-2, myb, MRG1, CREM, Alx4, FREAC1, NF-κB, members of the leucine zipper family, C2H4 zinc finger proteins, including Zif268, EGR1, EGR2, C6 zinc finger proteins, including the glucocorticoid and estrogen receptors. POU domain proteins, exemplified by Pit1, homeodomain proteins, including HOX-1, basic helix-loop-helix proteins, including myc, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor 1 (IRF-1). Wilms tumor protein, ETS-binding protein, STAT, GATA-box binding proteins, e.g., GATA-3, and the forkhead family of winged helix proteins.

Other useful gene products include carbamoyl synthetase 1, ornithine transcarbamylase, arginosuccinate synthetase, arginosuccinate lyase, arginase, fumarylacetoacetate hydrolase, phenylalanine hydroxylase, alpha-1 antitrypsin, glucose-6-phosphatase, low-density-lipoprotein receptor, porphobilinogen deaminase, factor VIII, factor IX, cystathione beta-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, beta-glucosidase, pyruvate carboxylase, hepatic phosphorylase, phosphorylase kinase, glycine decarboxylase (also referred to as P-protein), H-protein, T-protein, Menkes disease protein, tumor suppressors (e.g., p53), cystic fibrosis-transmembrane regulator (CFTR), and the product of Wilson's disease gene PWD.

Other useful transgenes include non-naturally occurring polypeptides, such as chimeric or hybrid polypeptides or polypeptides having a non-naturally occurring amino acid sequence containing insertions, deletions or amino acid substitutions. For example, single-chain engineered immunoglobulins could be useful in certain immunocompromised patients. Other types of non-naturally occurring gene sequences include antisense molecules and catalytic nucleic acids, such as ribozymes, which could be used to reduce overexpression of a gene.

The selection of the transgene sequence, or other molecule carried by lentiviral minigene, is not a limitation of this invention. Choice of a transgene sequence is within the skill of the artisan in accordance with the teachings of this application.

3. Regulatory Elements

Design of a transgene or another nucleic acid sequence that requires transcription, translation and/or expression to obtain the desired gene product in cells and hosts may include appropriate sequences that are operably linked to the coding sequences of interest to promote expression of the encoded product. “Operably linked” sequences include both expression control sequences that are contiguous with the nucleic acid sequences of interest and expression control sequences that act in trans or at a distance to control the nucleic acid sequences of interest.

Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. A great number of expression control sequences—natives constitutive, inducible and/or tissue-specific—are known in the art and may be utilized to drive expression of the gene, depending upon the type of expression desired. For eukaryotic cells, expression control sequences typically include a promoter, an enhancer, such as one derived from an immunoglobulin gene, SV40, cytomegalovirus, etc., and a polyadenylation sequence which may include splice donor and acceptor sites. The polyadenylation (polyA) sequence generally is inserted following the transgene sequences and before the 3′ lentivirus LTR sequence. Most suitably, the lentiviral minigene carrying the transgene or other molecule contains the polyA from the lentivirus providing the LTR sequences, e.g., HIV. However, other source of polyA may be readily selected for inclusion in the construct of the invention. In one embodiment, the bovine growth hormone polyA is selected. A lentiviral minigene of the present invention may also contain an intron, desirably located between the promoter/enhancer sequence and the transgene. One possible intron sequence is also derived from SV-40, and is referred to as the SV-40 T intron sequence. Another element that may be used in the vector is an internal ribosome entry site (IRES). An IRES sequence is used to produce more than one polypeptide from a single gene transcript. An IRES sequence would be used to produce a protein that contains more than one polypeptide chain. Selection of these and other common vector elements are conventional and many such sequences are available (see, e.g., Sambrook et al, and references cited therein at, for example, panes 3.18-3.26 and 16.17-16.27 and Ausubel et al., CURRENT PROTOCOLS IN MOLECULAR BIOLOGY. John Wiley & Sons, New York. 1989).

In one embodiment, high-level constitutive expression will be desired. Examples of useful constitutive promoters include, without limitation, the retroviral Rous sarcoma virus (RSV) LTR promoter (optionally with the RSV enhancer), the cytomegalovirus (CMV) promoter (optionally with the CMV enhancer) (see, e.g. Boshart et al, Cell, 41:521-530 (1985)), the SV40 promoter, the dihydrofolate reductase promoter, the β-actin promoter, the phosphoglycerol kinase (PGK) promoter, and the EF1α promoter (Invitrogen). Inducible promoters, regulated by exogenously supplied compounds, are also useful and include, the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al. Proc. Natl. Acad. Sci. USA, 93:3346-3351 (1996)), the tetracycline-repressible system (Gossen et al, Proc. Natl. Acad Sci. USA, 89:5547-5551 (1992)), the tetracycline-inducible system (Gossen et al, Science. 268:1766-1769 (1995), see also Harvey et al, Curr. Opin. Chem. Biol., 2:512-518 (1998)), the RU486-inducible system (Wang et al, Nat. Biotech., 15:239-243 (1997) and Wang et al, Gene Ther., 4:432-441 (1997)) and the rapamycin-inducible system (Magari et al, J. Clin. Invest., 100:2865-2872 (1997)). Other types of inducible promoters which may be useful in this context are those which are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

Another embodiment of the transgene includes a transgene operably linked to a tissue-specific promoter. For instance, if expression in skeletal muscle is desired, a promoter active in muscle should be used. These include the promoters from genes encoding skeletal α-actin, myosin light chain 2A, dystrophin, muscle creatine kinase, as well as synthetic muscle promoters with activities higher than naturally-occurring promoters (see Li et al., Nat. Biotech., 17:241-245 (1999)). Examples of promoters that are tissue-specific are known for liver (albumin, Miyatake et al. J. Virol. 71:5124-32 (1997); hepatitis B virus core promoter, Sandig et al., Gene Ther., 3:1002-9 (1996); alpha-fetoprotein (AFP). Arbuthnot et al., Hum. Gene Ther., 7:1503-14 (1996)), bone osteocalcin (Stein et al., Mol. Biol. Rep., 24:185-96 (1997)), bone sialoprotein (Chen et al., J. Bone Miner. Rep., 11:654-64 (1996)), lymphocytes (CD2, Hansal et al., J. Immumnol., 161:1063-8 (1998); immunogllobulin heavy chain; T cell receptor a chain), neuronal such as neuron-specific enolase (NSE) promoter (Andersen et al., Cell. Mol. Neurobiol., 13:503-15 (1993)), neurofilament light-chain gene (Piccioli et al., Proc. Natl. Acad. Sci. USA, 88:5611-5 (1991)), and the neuron-specific vgf gene (Piccioli et al., Neuron. 15:373-84 (1995)), among others.

Of course, not all expression control sequences will function equally well to express all of the transgenes of this invention. However, one of skill in the art may make a selection among these expression control sequences without departing from the scope of this invention. Suitable promoter/enhancer sequences may be selected by one of skill in the art using the guidance provided by this application. Such selection is a routine matter and is not a limitation of the molecule or construct. For instance, one may select one or more expression control sequences may be operably linked to the coding sequence of interest, and inserted into the transgene, the minigene, and the transfer virus of the invention. After following one of the methods for packaging the lentivirus minigene taught in this specification, or as taught in the art, one may infect suitable cells in vitro or in vivo. The number of copies of the minigene in the cell may be monitored by Southern blotting or quantitative RT-PCR. The level of RNA expression may be monitored by Northern blotting or quantitative RT-PCR. The level of expression may be monitored by Western blotting, immunohistochemistry, ELISA, RIA, or tests of the gene product's biological activity. Thus, one may easily assay whether a particular expression control sequence is suitable for a specific produced encoded by the transgene, and choose the expression control sequence most appropriate. Alternatively, where the molecule for delivery does not require expression, e.g., a carbohydrate, polypeptide, peptide, etc., the expression control sequences need not form part of the lentiviral minigene or other molecule.

4. Other Lentiviral Elements

Optionally, the lentivirus minigene may contain other lentiviral elements, such as are well known in the art, many of which are described below in connection with the lentiviral packaging sequences. However, notably, the lentivirus minigene lacks the ability to assemble lentiviral envelope protein. Such a lentivirus minigene may contain a portion of the envelope sequences corresponding to the RRE, but lack the other envelope sequences. However, more desirably, the lentivirus minigene lacks the sequences encoding any functional lentiviral envelope protein in order to substantially eliminate the possibility of a recombination event which results in replication competent virus.

Thus, the lentiviral minigene of the invention contains, at a minimum, lentivirus 5′ long terminal repeat (LTR) sequences, (optionally) a psi encapsidation sequence, a molecule for delivery to the host cells, and a functional portion of the lentivirus 3′ LTR sequences. Desirably, the minigene further contains RRE sequences or their functional equivalent. Suitably, a lentiviral minigene of the invention is delivered to a host cell for packaging into a virus by any suitable means, e.g., by transfection of the “naked” DNA molecule comprising the lentiviral minigene or by a vector which may contain other lentiviral and regulatory elements described above, as well as any other elements commonly found on vectors. A “vector” can be any suitable vehicle which is capable of delivering the sequences or molecules carried thereon to a cell. For example, the vector may be readily selected from among, without limitation, a plasmid, phage, transposon, cosmid, virus, etc. Plasmids are particularly desirable for use in the invention. The selected vector may be delivered by any suitable method, including transfection, electroporation, liposome delivery, membrane fusion techniques, high velocity DNA-coated pellets, viral infection and protoplast fusion.

According to the present invention, the lentiviral minigene is packaged in a heterologous (i.e., non-lentiviral) envelope using the methods described in part II below to form the transfer virus of the invention.

B. Envelope Protein

The envelope in which the lentiviral minigene is packaged is suitably free of lentiviral envelope protein and contains at least the binding domain of a filovirus envelope protein anchored to a membrane domain of a non-lentiviral envelope protein. The envelope may be derived entirely from filovirus olycoprotein, or may contain a fragment of the filovirus envelope (a filovirus polypeptide or peptide) which contains the binding domain fused in frame to a envelope protein, polypeptide, or peptide, of a second virus.

1. Filovirus Elements of Envelope

The filovirus which provides the sequences encoding the envelope protein or a polypeptide or peptide thereof (e.g., the binding domain) can be derived from an Ebola virus, selected from any suitable serotype, e.g., Zaire. Sudan, or Reston. Alternatively, another filovirus envelope protein may be utilized, e.g., a envelope protein from Marburg virus. The sequences encoding the envelope protein may be obtained by any suitable means, including application of genetic engineering techniques to a viral source, chemical synthesis techniques, recombinant production, or combinations thereof. Suitable sources of the desired viral sequences are well known in the art, and include a variety of academic, non-profit, commercial sources, and from electronic databases. The methods by which the sequences are obtained is not a limitation of the present invention.

In one desirable embodiment, the filovirus envelope sequences are derived from the Ebola virus, most preferably Zaire strain, glycoprotein. In filoviruses, the glycoprotein gene is the fourth gene (of seven) from the 3′ end of the negative strand RNA genome. Thus, each of the filoviruses contains a type of glycoprotein organization in which a secreted, non-structural protein is expressed in preference to the structural glycoprotein. In the Ebola Zaire strain, the secreted glycoprotein is 50 to 70 kD, and the type 1 transmembrane form encodes a 120- to 150-kD glycoprotein that is incorporated into the virion. The first 295 amino acids of both proteins are identical in the Zaire strain, but the secreted glycoprotein (sGP) contains an additional 60 amino acids and the transmembrane glycoprotein (GP) contains another 381 COOH-terminal amino acid residues (A. Sanchez et al., J. Virol., 72(8):6442-6447 (1998)). As these two glycoproteins are known to target different cell types, either may be selected, depending upon the target cell. However, as sGP binds to neutrophils, it may not be as desirable as GP, which binds to endothelial cells. Similar structural glycoproteins may be readily obtained from the other Ebola viral strains, or from Marburg virus glycoprotein, which has been described in comparison to the Ebola virus genome (A. Sanchez et al, Virus. Res., 29(3):215-240 (September 1993)).

Thus, in one embodiment, the envelope protein is intact filovirus glycoprotein. Alternatively, it may be desirable to utilize a fragment of the selected filovirus which contains, at a minimum, the binding domain of the filovirus envelope glycoprotein, which is located within about amino acid 180 to amino acid 350 of the Ebola Zaire strain. Suitably, this filovirus protein fragment is fused, directly or indirectly, via a linker, to a second, non-lentiviral, envelope protein or fragment thereof. This fusion protein may be desirable to improve packaging, yield, and/or purification of the resulting envelope protein. The second, non-lentiviral envelope protein or fragment thereof contains, at a minimum, the membrane domain. In one desirable embodiment, a truncated fragment of the Ebola envelope protein (deleted of aa 649-676, i.e., the C-terminal 27 aa of the envelope) is fused to the last 48 amino acids of the VSVG envelope protein (aa 448 to 496). Still other fusion (chimeric) proteins according to the present invention can be generated by one of skill in the art.

2. Chimeric Envelope Glycoproteins

Thus, in one embodiment, a useful envelope may be a chimeric glycoprotein containing the binding domain of a filovirus envelope glycoprotein fused to a fragment of a second envelope glycoprotein or a non-contiguous fragment of a filovirus capsid protein. For example, a selected filovirus binding domain may be fused to a filovirus transmembrane domain of the same or another selected filosvirus or filovirus strain. In Ebola Zaire strain, the transmembrane domain corresponds to residues 650 to 672 of the full-length glycoprotein (676 total residues). Similar regions may be obtained from the another filovirus.

In another embodiment, the second protein or fragment may be derived from another non-lentiviral source. For example, one suitable envelope protein may contain the membrane domain from vesicular stomatitis virus (VSV) glycoprotein (G). Alternatively, other suitable fragment may be selected from another suitable viral source which provides the desired packaging levels. The present invention is not limited by the selection of the source of this non-lentiviral membrane domain fragment, which may be readily selected by one of skill in the art taking into consideration such factors as, the system in which the lentiviral minigenes are to be packaged, including the expression vectors utilized and the host cell used for packaging, as well as the type(s) of cells to which the lentiviral minigene will be delivering the transgene, as well as the type of molecule to be delivered.

Where the envelope is a fusion protein, a linker may be inserted between the sequences encoding the filovirus envelope protein (or fragment thereof) and the sequences encoding the second envelope protein (or fragment thereof). Such a linker may desirable, in order to ensure that, upon expression, an envelope which is a fusion protein is produced. Thus, the linker may be a spacer which ensures that the two sequences are appropriately translated. Such a linker may be nucleic acids (preferably non-coding sequences) or it may be a chemical compound or other suitable moiety. Suitable techniques for designing such a fusion protein are well known to those of skill in the art. See, generally. Sambriolok et al, Molecular Cloning: A Laboratory Manual, Cold Spring Harbor Laboratories, Cold Spring Harbor, N.Y.

Methods of producing the transfer viruses of the invention using packaging host cells are described below.

### II. Production of Recombinant Transfer Virus

Thus, the invention further involves a method of producing a recombinant transfer virus useful for delivering a selected molecule to a host cell.

In one embodiment, in vitro packaging techniques may be utilized, in which the envelope protein is produced in a host cell using the techniques described herein, extracted using conventional protein extraction techniques, and used to package the virus in vitro.

In another embodiment, this method involves the steps of culturing in a host cell lentivirus packaging sequences, a lentiviral minigene carrying the transgene or other molecule, and a vector encoding the envelope protein. Any or all of the components described herein for use in packaging the recombinant transfer virus of the invention may be provided in a stable cell line containing the desired component, e.g., the lentivirus packaging sequences, the lentiviral minigenie, or the nucleic acid sequences encoding the envelope protein. Alternatively, the lentivirus packaging sequences, the lentiviral minigene and/or the sequences encoding the envelope protein may be provided in trans.

As used herein, a transfer vector carries the lentiviral elements described in part I above, on a vector which delivers the lentiviral minigene to a cell in which it is to be packaged in the envelope protein. Optionally, the lentiviral packaging vector may also be the transfer vector. More preferably, this lentivirus minigene is supplied separately from the packaging sequences. In one suitable embodiment, the transfer virus is produced using a three vector system in which the host cell is separately provided with a nucleic acid molecule providing the lentivirus packaging sequences, a vector encoding the envelope protein, and a transfer vector which carries the lentiviral minigene which will be packaged into the envelope protein. In still another alternative, a host cell may be utilized which has been engineered to stably or inducibly express one or more of the lentivirus packaging sequences, the lentiviral minigene, or nucleic acid sequences expressing the envelope protein, thus avoiding a separate transfection technique. Such a host cell may be designed and produced using techniques which are known to those of skill in the art. In any of these alternatives, following transfection/infection, the host cell is cultured under conditions which permit packaging of the lentivirus minigene carrying the molecule for delivery in the envelope protein.

A. Lentiviral Packaging Sequences

In order to package the lentivirus minigene in the envelope containing filovirus binding domain, the host cell must be provided with lentiviral packaging sequences. At a minimum, the lentiviral packaging sequences required are those responsible for gag and gag-pol polyprotein expression. Suitably, these sequences include gag and pol genes operably linked to sequences which direct their expression and nuclear localization. Optionally, these sequences may contain a sufficient amount of the RRE to provide the desired function, e.g., about 400 nt of the RRE, as discussed above.

Suitably, the lentiviral packaging sequences may be obtained from any lentiviral source, as described above in Part I for the lentiviral minigene. Optionally, the lentiviral sequences in the packaging vector may be derived from the same lentivirus as the lentivirus minigene, or from a lentivirus source which is cross-reactive with the lentivirus sequences in the minigene. For example, the packaging sequences may be obtained from FIV or SIV, where the lentiviral sequences of the minigene are obtained from HIV. Selection of the lentiviral sequences in the packaging vector is not a limitation of the present invention.

These lentiviral sequences may be provided to the host cell by any suitable means. In one embodiment, the sequences are supplied on a single vector, i.e., a lentiviral packaging vector delivers to the packaging host cell at least the minimal sequences described above. This vector can be any suitable vehicle which is capable of delivering the lentiviral packaging sequences to the selected host. For example, the vector may be readily selected from among any suitable genetic element from which can be expressed the elements required for lentiviral packaging, including, without limitation, a plasmid, phage, transposon, cosmic, virus, virion. However, plasmids are particularly desirable for use in the method of the invention.

In order to be useful for packaging the lentiviral minigene in the heterologous envelope protein provided to the host cell, the packaging vector lacks the ability to express functional lentivirus envelope proteins. In one embodiment, the vector contains a deletion in a portion of the envelope protein. An example of such a deletion is described in L. Naldini et al, Science, 272:263-267 (Apr. 12, 1996). More desirably, however, the construct lacks the sequences encoding functional envelope proteins. Thus, the packaging vector may have a partial deletion of envelope sequences, or a deletion of the entire region encoding the envelope proteins. Regardless of the extent of the deletion, no functional lentiviral envelope proteins are expressed.

Additionally, in order to minimize the possibility of a replication-competent recombinant event, it is preferable to replace the native 5′ LTRs of the lentivirus with a heterologous promoter which drives expression of the gag and pol proteins. The promoter may be readily selected from among the promoters identified herein, and is preferably a strong promoter. Similarly, it is preferable for the native 3′ LTRs of the lentivirus to be substituted with a polya sequence, which may be derived from any suitable source.

The lentiviral packaging vector may also contain other desirable vector elements, including splice donor (SD) sites (such as the SD site located upstream of the ψ site which native to the lentiviruses), the splice acceptor sites, RRE sequences and the like. The selection and inclusion of such vector elements is not a limitation of the present invention.

The lentiviral packaging vector may contain other bicistronic lentiviral elements, such as the tat, vif, vpr, vpu and nef sequences, however, these sequences are not required for packaging and are more preferably eliminated. Preferably, the lentivirus packaging sequences and the lentiviral minigene are supplied to the packaging cell separately.

In another embodiment, the lentiviral packaging sequences may be supplied to the host cell separately, for example, by use of separate vectors or by providing a host cell which expresses one or more of the required lentiviral packaging elements (e.g., gag or pol). In still another embodiment, the host cell expresses all required lentiviral packaging elements.

B. Nucleic Acid Molecule Encoding Envelope Protein

The nucleic acid molecule carrying the sequences encoding the envelope protein operably linked to its expression control sequences as described above, may be readily selected from among any suitable genetic element (i.e., vector) from which the envelope protein can be expressed in the host cell. However, a plasmid is preferred for this purpose. A suitable expression plasmid may be readily selected by one of skill in the art taking into consideration convenience, the selected expression cells, and the like.

The necessary envelope protein sequences and regulatory elements may be readily engineered into the selected vector. The envelope sequences are readily selected from a variety of sources identified above. The regulatory sequences may be readily selected from among the sequences described above in the section discussing regulatory sequences for the transgene. Thus, the nucleic acid molecule carrying the envelope protein contains the envelope sequences described above under the control of regulatory sequences which direct expression of the envelope protein in a host cell.

C. Packaging Cell, Production, Purification

Conventional techniques may be utilized for construction of the lentiviral minigenes and other nucleic acid molecules of the invention. See, generally. Sambrook et al, MOLECULAR CLONING: A LABORATORY MANUAL, Cold Spring Harbor Laboratories, Cold Spring Harbor, N.Y. Once the desired vectors are engineered, they may be transferred to a host cell for packaging into the viral envelope by any suitable method. Such methods include, for example, transfection, electroporation, liposome delivery, membrane fusion techniques, high velocity DNA-coated pellets, viral infection and protoplast fusion.

The host cell itself may be selected from any prokaryotic cell, including any bacteria cell, or any eukaryotic cell, including insects, and yeast, among others. In one desirable embodiment, the host cell is selected from among mammalian species, and particularly from among human cell types. Suitable cells include, without limitation, cells such as CHO, BKH, MDCK, and various murine cells, e.g., 10T1/2 and WEHI cells, African green monkey cells, suitable primate cells, e.g., VERO, COS1, COS7, BSC1, BSC 40, and BMT 10, and human cells such as W138, MRC5, A549, human embryonic retinoblast (HER), human embryonic kidney (HEK), human embryonic lung (HEL), TH1080 cells. Other suitable cells may include NIH3T3 cells (subline of 3T3 cells). HepG2 cells (human liver carcinoma cell line). Saos-2 cells (human osteogenic sarcomas cell line), HuH7 cells or HeLa cells (human carcinoma cell line). In a preferred embodiment, appropriate cells include the human embryonic kidney 293T cells (which express the large T antigen) (ATCC). Neither the selection of the mammalian species providing the cells nor the type of mammalian cell is a limitation of this invention.

Regardless of whether a double transfection or triple transfection technique is utilized, the host cells are cultured according to standard methods. See, e.g., R. J. Wool-Lewis and P. Bates, J. Virol. 74(4):3155-3160 (April 1998): see, also. Sambrook et al, cited above.

As discussed above, a host cell which stably contains one or more of the desired elements, e.g., gag, pol, the psi sequences, the RRE (or functionally equivalent) sequences, the lentiviral minigene, and/or the envelope protein, may be prepared using techniques known to those of skill in the art. Such techniques include cDNA, genomic cloning, which is well known and is described in Sambrook et al, cited above, and use of overlapping oligonucleotides in the target sequences, combined with polymerase chain reaction, synthetic methods, and any other suitable methods which provide the desired nucleotide sequence. Introduction of the molecules (as plasmids or another vector element) into the host cell may also be accomplished using techniques known to the skilled artisan and as discussed throughout the specification. In a preferred embodiment, standard transfection techniques are used, e.g., CaPO₄ transfection, transfection using the Effectene™ reagent, or electroporation, and/or infection by viral vectors into cell lines such as those described above. Each of the desired sequences stably contained within the host cell may be under the control of regulatory elements, such as those discussed above in connection with the transgene. In one particularly suitable embodiment, inducible promoters are selected. For example, it may be particularly desirable for gag and pol to be expressed under the control of one or more inducible promoters. However, other suitable regulatory elements may be readily selected by one of skill in the art.

In another embodiment, it may be particularly desirable for the host cell to be provided with enzymatic genes which are useful or necessary for packaging of the lentivirus in the heterologous envelope. One particularly suitable enzyme is the lentiviral protease, which is necessary for processing of the gag/pol. However, other enzymes such as integrase, reverse transcriptase, and/or other non-lentiviral enzymes which provide equivalent enzymatic functions may be readily selected. The separation of these genes from the constructs used to deliver the other lentiviral elements to the host cell adds a further safeguard against the possibility of a homologous recombination event in the host cell.

Regardless of the production method utilized, the recombinant transfer viruses of the invention may be readily purified from culture using methods known to those of skill in the art. One suitable method involves ultracentrifugation with or without sucrose or affinity chromatography. Conventional techniques may be used to concentrate the recombinant transfer virus (see, e.g., J. C. Burn et al. Proc. Natl. Acad. Sci. USA, 90:8033-8037 (1993)).

### III. Pharmaceutical Compositions

The transfer viruses according to the present invention are suitable for a variety of uses including in vitro protein and peptide expression, as well as ex vivo and in vivo gene delivery.

The recombinant transfer viruses of the invention may be used to deliver a selected transgene or other molecule to a host cell by any suitable means. In one embodiment, the transfer viruses and the cells are mixed ex vivo and the infected cells are cultured using conventional methodologies. Such methods are described in more detail below.

Alternatively, the recombinant transfer viruses, preferably suspended in a physiologically compatible carrier, may be administered to a human or non-human mammalian patient. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention.

Optionally, the compositions of the invention may contain, in addition to the recombinant transfer virus and carrier(s), other conventional pharmaceutical ingredients, such as preservatives, chemical stabilizers, or for vaccine use, adjuvants. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin. Suitable exemplary adjuvants include, among others, immune-stimulating complexes (ISCOMS), LPS analogs including 3-O-deacylated monophosphoryl lipid A (Ribi Immunochem Research, Inc.; Hamilton, Mont.), mineral oil and water, aluminum hydroxide. Amphigen, Avirdine. L121/squalene, muramyl peptides, and saponins, such as Quil A.

In one embodiment, transfer viruses have been deemed suitable for applications in which delivery of a molecule, e.g., a transgene which permits transient expression, is therapeutic (e.g., p53 gene transfer in cancer and VEGF gene transfer in heart diseases). However, the transfer viruses are not limited to use where transient expression is desired. The transfer viruses are useful for a variety of situations in which delivery of a selected molecule is desired.

Thus, the recombinant transfer viruses of the invention, are useful for any of the variety of gene or non-gene delivery applications. However, these recombinant transfer viruses of the invention provide significant advantages over prior art viruses.

### IV. Therapeutic Methods

Thus, the invention provides a method of delivering a transgene or other molecule to a human or veterinary patient by transducing the cells of the patient with a recombinant transfer virus according to the invention. The target cells may be transduced in vivo or ex vivo, taking into consideration such factors as the selection of target cells, the transgene being delivered, and the condition for which the patient is being treated. For example, where the targeted cells are selected from muscle cells, lung cells, liver cells or the like, in vivo transduction may be more desirable. However, where the targeted cells are dendritic cells and/or macrophages, ex vivo transduction is preferred.

A. In vivo

For in vivo delivery of the transgenes, any suitable route of administration may be used, including, direct delivery to the target organ, tissue or site, intranasal, intravenous, intramuscular, subcutaneous, intradermal, vaginal, rectal, and oral administration. Routes of administration may be combined within the course of repeated therapy or immunization.

Advantageously, the transfer viruses of the invention are particularly well suited to delivery of transgenes and other molecules to lung cells, as these viruses infect from the apical site, and thus are suited to intratracheal, intranasal, aerosol [Penn Century Sprayer Device, Penn Century, Philadelphia, Pa.; U.S. Pat. No. 5.579.578] or other suitable delivery means. Although less desirable, bronchoscopy may also be utilized for delivery. In one particularly desirable embodiment, the transfer virus of the invention is engineered to contain a cystic fibrosis transmembrane conductance regulator (CFTR) gene which is delivered intratracheally. In another embodiment, it may be desirable to treat a solid tumor by injection of a transfer virus carrying a selected transgene (e.g., IL-2. IL-12, TNF, GM-CSF, herpes simplex virus thymidine-kinase (HS-tk), telomerase, a toxic molecule, a suicide gene, or the like), directly into the tumor. However, the invention is not limited as to selection of transgene or other molecule, or route of delivery, as discussed above.

Suitable doses of transfer viruses may be readily determined by one of skill in the art, depending upon the condition being treated, the health, age and weight of the veterinary or human patient, and other related factors. However, generally, a suitable dose may be in the range of 10³ to 10⁸, preferably about 10⁵ to 10¹⁶ transducing units (TU) per dose, and most preferably, about 10⁷ to 10⁹ TU for an adult human having a weight of about 80 kg. Transducing Units (TU) represents the number of infectious particles and is determined by evaluation of transgene (e.g., lacZ) expression upon infection of target cells (usually 293T cells) with limiting dilution of each virus preparation. This dose may be formulated in a pharmaceutical composition, as described above (e.g., suspended in about 0.01 mL to about 1 mL of a physiologically compatible carrier) and delivered by any suitable means. The dose may be repeated, as needed or desired, daily, weekly, monthly, or at other selected intervals.

B. Ex Vivo

In another embodiment, the transfer viruses of the invention are useful for ex vivo transduction of target cells. Generally, in vivo therapy involves removal of a population of cells containing the target cells, transduction of the cells in vitro, and then reinfusion of the transduced cells into the human or veterinary patient. Such ex vivo transduction is particularly desirable when the target cells are dendritic cells or macrophages and/or when the transgene or other molecule being delivered is highly toxic, e.g., in the case of some genes used in the treatment of cancer. However, one of skill in the art can readily select ex vivo therapy according to the invention, taking into consideration such factors as the type of target cells to be delivered, the molecule to be delivered, the condition being treated, the condition of the patient, and the like.

In one embodiment it may be desirable to treat a circulating cancer (e.g., leukemia or lymphoma) by ex vivo therapy, by removal of bone marrow cells or peripheral blood T lymphocytes, transduction with the transfer virus of the invention in vitro, and re-infusion of the transduced cells. In another embodiment, it may be desirable to treat a solid tumor by surgical removal of tumor cells, ex vivo transduction of dendritic cells with the transfer virus of the invention carrying an antigenic epitope from the excised tumor, and re-infusing the altered dendritic cells to induce specific immunity to the antigen. In yet another embodiment, it may be desirable to treat hypercholesterolemia or hyperlipidemia by removal of liver cells (hepatocytes), transduction of the cells with a transfer vector carrying the LDLr gene or VLDLr gene in culture, and re-infusion of these cells via the portal vein. Still other suitable conditions for ex vivo therapy and other useful transgenes will be apparent to one of skill in the art.

Generally, when used for ex vivo therapy, the targeted host cells are infected with 10⁵ TU to 10¹⁰ TU transfer viruses for each 10¹ to 10¹⁰ cells in a population of target cells. However, other suitable ex vivo dosing levels may be readily selected by one of skill in the art.

The following examples are provided to illustrate construction and use of the recombinant vectors and compositions of the invention and do not limit the scope thereof. One skilled in the art will appreciate that although specific elements, reagents and conditions are outlined in the following examples, modifications can be made which are meant to be encompassed by the spirit and scope of the invention.

### EXAMPLE 1

### Production of Vectors for Production of Pseudotyped HIV/Ebola Viruses

A. Plasmid Encoding Ebola Zaire Strain Envelope

The plasmid, pCB-Ebo-GP was constructed using the techniques described in R. J. Wool-Lewis and P. Bates, J. Virol., 74(4):3 155-3160 (April 1998). Briefly, the cDNA encoding the Zaire subtype of Ebo-GP was obtained from the Centers for Disease Control and Prevention in the vector pGEM3Zf(-) as a BamHI-KpnI fragment. The Ebo-GP gene was excised from pGEM3Zf(-), using the BamHI and EcoRI restriction enzymes, and cloned into an mammalian expression plasmid pCB6 (purchased commercially) downstream of a human cytomegalovirus promoter to create the plasmid pCB6-Ebo-GP.

B. Lentiviral Packaging Sequences

The HIV-1 packaging vector, pCMVΔR8.2, was constructed as follows, pR8 is a plasmid containing the sequences of an infectious molecular clone of HIV-1. A 39 bp deletion was made in the packaging signal (ψ) sequence of the HIV genome by digestion of pR8 with BssH-II and Spel. A NotI-Xbal cassette containing the polyA site was obtained from human insulin genomic DNA using standard polymerase chain techniques and engineered by PCR into the Xbal-NotI site at the end of the nef ORF. A heterologous CMV promoter was inserted in the place of the 5′ LTRs and a region of the HIV-1 envelope corresponding to bp 6307-7611 of the HIV-1 genome was deleted by digestion of the plasmid with NotI.

The resulting plasmid, pCMVΔR8.2, contains a human CMV promoter, a defective HIV-1 packaging signal (ψ) containing a 39 bp deletion, intact gag and pol, a deletion in the envelope region corresponding to the env promoter sequences, RRE, and a polyA sequence.

C. Transfer Vector Carrying Lentivirus Minigene with Marker Gene

The plasmid, pHR′CMVlacZ, was produced as described in L. Naldini et al, Science, 272:263-267 (Apr. 12, 1996). Plasmid HR′ was constructed by cloning a fragment of the HIV env gene compassing the RRE and a splice acceptor site between the two LTRs of the HIV-1 proviral DNA. The gag gene was truncated and its reading frame blocked by a frameshift mutation, pHR′-CMVLacZ was generated by cloning a 3.6-kbp SalI-Xhol fragment containing the CMV promoter and the E. coli lacZ gene (encoding β-galactosidase) from plasmid pSLX-CMVlacZ (R. Scharfmann, et al, Proc. Natl. Acad. Sci. USA, 88:4626 (1991)).

The resulting plasmid, pHR′-CMVLacZ, contains the 5′ LTRs, a splice donor site, the ψ packaging signal, the RRE sequences, a splice acceptor site, the CMV promoter, the LacZ transgene, and the 3′ LTRs. The lentiviral sequences in this plasmid are from HIV-1.

### EXAMPLE 2

### Production of Vectors for Production of Pseudotyped Lentiviruses

The helper packaging construct pCMVΔR8.2 encoding for the HIV helper function, the transfer vector pHR′LacZ encoding for the β-gal and plasmids encoding for envelope proteins were used for triple transfection.

The transfer vector pHR′EGFP was generated by cloning the BamHI/blunted Bell containing the EGFP ORF from pCMS-EGFP (Clontech, Palo Alto, Calif.) into the BamHI/blunted EcoRI site of pHR′LacZ.

Plasmids encoding the following viral envelopes were used to generate pseudotyped viruses: pMD.G (U. Blomer, et al., J. Virol., 71:6641-6649 (1997)) and pLTRMVG encoding for the Rhabdoviridae VSV-G and Mokola (H. Mochizukil et al, J. Virol. 72:8873-8883 (1998)) envelopes, pHIT 456 (R. Lodge, et al, Gene Ther., 5:655-664 (1998)) encoding for the Oncovirus Murine Leukemia Virus (MuLV) amphotropic envelope, pCB6-Ebo-GP (R. J. Wool-Lewis and P. Bates, J. Viorol., 72:3155-3160 (1998)) encoding for the Filovirus Ebola-Zaire (EboZ) envelope, pCB6-Ebo-GPR encoding for the Ebola-Reston (EboR) envelope, pSVCMVinHA encoding for the Orthomyxovirus Influenza-FIA envelope and pSVCMVinF and pSVCMVinG encoding for the Paramyxovirus Respiratory Syncitial Virus (RSV) F and G envelope proteins, pSVCMVinHA was engineered by cloning the blunted ClaI/Asp718 fragment containing the Influenza envelope from BH-RCANsHA (J. Dong, et al., J. Virol., 66:7374-7382 (1992)) into SVCMVin at the SmaI site. To construct pSVCMVInF and pSVCMVinG, genomic RNA was extracted from RSV virions (ATCC #VR-1401) using Trizol reagent (Gibco BRL, Rockville, Md.).

The F and G proteins were amplified by RT-PCR with the following mutagenic primers: F-sense SEQ ID NO: 1: 5′ AATTAAACCTGAAGCTTATAACCATGGAGC, F-antisense. SEQ ID NO: 2: 5′ GGTGATCAGCAGACGTCTGTTGAAACATG, G-sense. SEQ ID NO: 3: GGGATCAAAAA CAAGCTTGGGGCAAATGC and G-antisense. SEQ ID NO: 4: 5′ AAGATGTAGTTTGACGTCAA CTAAGCATG. The PCR amplified fragments were digested in HindIII/PstI and cloned into the corresponding site of SVCMVin. SVCMVin was derived from SVCMV (X. Yao, et al, Gene Ther., 6:1590-1599 (1999)) and contains an intron from the β-globin gene, the SV40 ORI, and a CMV promoter driving the transgene.

### EXAMPLE 3

### Production of Pseudotyped Virus

A. Production of FIIV-1 Miziiene Pseudotyped in Ebola Envelope

In one early experiment, pCB-Ebo-GP was mixed with the a plasmid encoding the packageable genome encoding a marker gene (pHR′CMVlacZ) and the plasmid encoding the lentiviral packaging sequences (pCMVΔR8.2). This DNA mixture was transfected into 293T cells by a standard CaPO₄ transfection procedure. Briefly, the 293T cells were seeded at between 50 and 70% confluence the day prior to transfection. The 293T cells were refed 1 hour prior to transfection. Then, a DNA cocktail containing between 20 and 60 μg of the DNA to be transfected. 50 μl of 10×NTE (8.77 g of NaCl, 10 ml of 1 M Tris [pH 7.4], and 4 mL of 0.25M EDTA [pH 8.0] in a final volume of 100 ml in H₂O), and 62.5 μl of 2 M CaCl₂, brought to a final volume of 500 μL with H₂O, was prepared. This DNA cocktail was added dropwise to 500 μL of 2× transfection buffer (1 mL of 0.5 M IIEPES [pH 7.1 ]. 8.1 mL of H₂O, 0.9 ml of 2 M NaCl, and 20 μl of 1 M Na₂HPO₄) and left at room temperature for 30 min. This solution was then added dropwise to the 293 T cells and left on overnight. The next day, the cells were refed with fresh medium.

Forty-eight hours posttransfection medium containing virus was collected and clarified by filtration through a 0.45 μm pore-size syringe filter. These supernatants were stored at either 4 or −80° C. as viral stocks. Transfected-cell monolayers were lysed and analyzed for Ebo-GP expression by Western blot analysis as described (L. Rong and P. Bastes, J. Virol. 69:4847-4853 (1995)) using an anti-Ebo-TP antibody at a 1:1000 dilution and a horseradish peroxidase-conjugated goat anti-rabbit secondary antibody (Pierce, Rockford, Ill.) at a 1:20,000 dilution.

B. Calcium Phosphate Transfection Procedure

For the experiments described below, pseudotyped virus was produced by triple transfection using either the CaPO₄ precipitation method (Clontech) or Effectene reagent (Qiagen, Valencia, Calif.). For both methods, envelope expression vector, HIV packaging plasmid encoding viral genes, and transfer vector encoding the transgene were mixed in a 3:1:2 molar ratio.

Using the CaPO₄ transfection procedure as described by the manufacturer (Clontech), 10 μg or 180 μg of endotoxin free DNA mixture was applied to each 60 mm or 150 mm plate of 293 T cells, respectively.

C. Transfection Using Effectene Reagent

Transfection using the Effectene reagent was performed according to manufacturer's guidelines with adjustments for the amount of lipid (40 μl for 60 mm and 2.9 ml for 150 mm plate), EC buffer (800 μl for 60 mm and 58 ml for 150 mm plate) and enhancer (55 μl for 60 mm and 4 ml for 150 mm plate) for 10 μg or 180 μg of the DNA mixture per 60 mm or 150 mm plate respectively. 44 h after transfection, media was added to each plate for 16 h prior to collection of virus. The media containing virus-like particles was filtered through a 0.45 μm filter and used to transduce target cell lines by using limiting dilution for titering. Cell free supernatant containing virus was also concentrated by ultracentrifugation at 28K rpm for 2 h at 4° C. by using a SW28 rotor (Beckman, Fullerton, Calif.). Virus was resuspended in complete DMEM and stored at −80° C. Several highly concentrated pseudotyped viral stocks were tested for the presence of Replication Competent Lentivirus by monitoring p24 antigen expression (T. Dull, et al., J. Virol., 72:8463-8471 (1998)) in the culture medium of transduced MT4 and 293T cells for 30 days. In all cases tested, p24 was undetectable once the input antigen had been eliminated from the culture.

C. Generation of Virus Stock

Virus stock for screening of different envelope pseudotypes was generated by ultracentrifugation of viral supernatant harvested from ten 60 mm transfected plates of each envelope pseudotyped virus. Vector was then resuspended in of complete DMEM and applied to ALI cultures. For high titer application, the vector was generated by ultracentrifugation of viral supernatant from twenty 150 mm transfected plates of each envelope pseudotyped virus. Each vector was resuspended in 200 μl of complete DMEM, generating highly concentrated viral stocks, and applied to the apical surface of ALI cultures.

All experiments involving the production and functional analysis of replication incompetent HIV-based pseudotyped vectors were performed under biosafety level 3 containment as approved by the Wistar Institute Institutional Biosafety Committee.

D. Creation Pseudotyped Lentiviral Vectors

The following viral envelopes were used for pseudotyping: Murine Leukemia Virus (MuLV) amphotropic envelope. Mokola envelope, Ebola-Zaire (EboZ) envelope. Ebola-Reston (EboR) envelope, Influenza-HA envelope and Respiratory Syncitial Virus (RSV) F and G envelope proteins. All pseudotyped viruses were produced in parallel under the same conditions for every experiment. Since each viral envelope protein used to pseudotype vector conferred specific tropism to the vector, titers established by limiting dilution on target cell lines were different and thus not used for normalizing the amount of input vector (Table 1). Consequently, each transduction was performed by using the same volume of concentrated vector produced from the same amount of cells transfected under the same conditions. Stocks were assayed for reverse transcriptase (RT) activity as previously described (G. P. Kobinger, et al., J. Virol., 72:5441-5448 (1998)), viral stocks had an average activity of 2×10⁵ counts/min/μl. Pseudotyped vectors applied on tissues for transduction demonstrated similar RT activity indicating that comparable amounts of virus-like particles were used (data not shown).

TABLE 1
Titers of stocks of different pseudotyped HIV vectors

| | 293 T | HeLa | MDCK |
|---|---|---|---|
| Ebo-Z | 3.75 × 10⁵ | 2.8 × 10⁵ | 6.1 × 10⁵ |
| Ebo-R | 4.8 × 10⁵ | 3.0 × 10⁴ | 8.5 × 10⁴ |
| VSVG | 3.1 × 10⁷ | 2.2 × 10⁷ | 5.1 × 10⁶ |
| Mokola | 3.1 × 10⁶ | 7.2 × 10⁵ | 1.7 × 10⁶ |
| HA | Not detected | 1.6 × 10² | Not detected |
| MoMuLV | 2.0 × 10⁶ | 2.9 × 10⁶ | 1.7 × 10⁵ |
| RSV | Not detected | 2.1 × 10³ | Not detected |
| Env (−) | Not detected | Not detected | Not detected |

Limiting dilutions were established with filtered. unconcentrated vector stocks produced from triple transfected 293T cells. Titers reflect the number of GFP transducing units/ml. Values represent mean of 2 titering experiments which gave similar results.

All of the screened pseudotyped viruses were shown to transduce, to varying degrees, a panel of target cell lines, suggesting that the envelopes were incorporated into the virions (Table 1). HA-pseudotyped HIV particles were able to promote agglutination of erytlirocytes in vitro, providing further evidence that this envelope was packaged despite low titers (data not shown). Surprisingly, envelopes from the influenza virus and RSV did not promote efficient transduction of human airway cells by HIV-based vector although these viruses commonly cause severe lung infections. Preliminary data indicates that both the RSV and HA envelopes may be damaged or shed during ultracentrifugation, rendering concentrated stocks of these viruses only slightly more infectious than unconcentrated stock. Indeed, RSV pseudotyped vector increased titer only 1.6 fold for a 100 fold volume concentration in contrast to EboZ pseudotyped vector increasing titer 75 fold for the same volume concentration (data not shown). It is possible that modification of wild type envelopes, such as deletion or addition of a domain derived from envelope proteins of other viruses, might increase stability of the pseudotyped vector. Therefore, in a different context, stocks of vectors pseudotyped with proteins derived from RSV or HA envelopes might be generated in high titers to promote efficient transduction of airway epithelia.

### EXAMPLE 4

### Identification of Viral Envelopes that Mediate Apical Transduction of Human Airway

Viruses pseudotyped with a variety of envelopes were applied to air-liquid interface (AlI) cultures of airway epithelial cells apically or basolaterally and analyzed 4 days later for GFP expression, as follows.

Human airway cells were digested from airways of explanted lungs from patients undergoing lung transplantation and seeded onto collagen coated permeable supports (Corning, Cambridge, Mass.) using previously published methods and used to establish air-liquid interface (ALI) cultures (G. Wang, et al, J. Virol., 72:9818-9826 (1998); J. F. Engelhardt, et al, Nat Genet., 4:27-34 (1993)). ALI cultures were maintained for approximately 14 days and until an adequate transepithelial resistance (>500 ohms×cm²) was generated, and no defects in the membrane could be visualized using light microscopy. For screening of viral envelopes. ALI cultures were transduced with partially concentrated (50 μl, concentrated 100 fold) GFP-encoding viruses applied from the apical or basolateral side. Transepithelial resistance was measured 24 h after infection and remained above 500 ohms×cm² (data not shown), suggesting that the epithelial integrity was not compromised. Cultures were examined using fluorescent microscopy at 4 days following transduction, and GFP expressing cells were counted by examining 20 fields at 100× magnification and extrapolating for the surface area of the support.

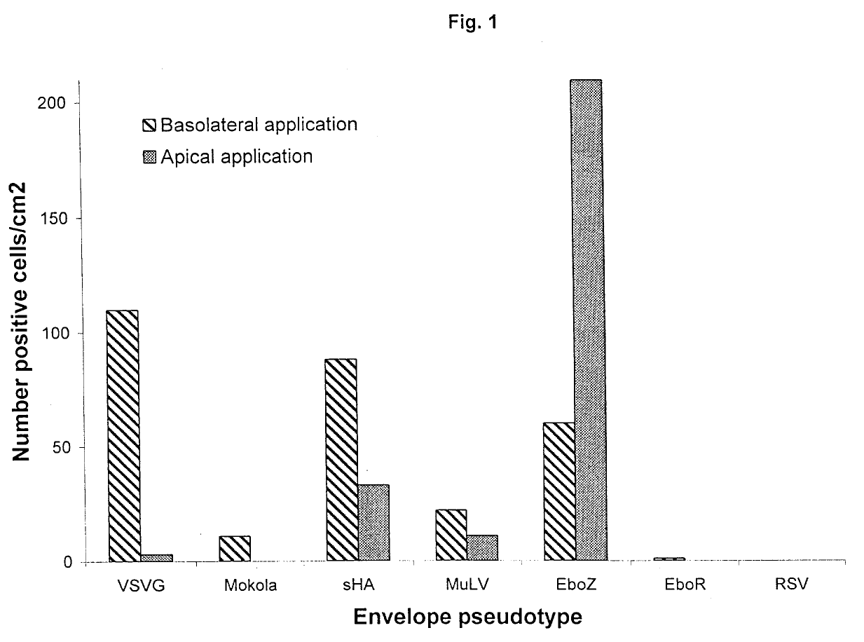

Following basolateral application, the panel of pseudotyped HIV vectors transduced between 0 and 110 cells/cm². In contrast, apical application resulted in poor transduction by all pseudotyped vectors with the exception of EboZ-pseudotyped virus for which more than 200 positive cells/cm² were detected. These experiments demonstrated the relative efficiency of EboZ-pseudotyped virus compared to other pseudotyped vectors. However, the overall transduction efficiency is less than 0.1% of all cells, and therefore may not attain a level that is clinically relevant using partially concentrated vector stocks, FIG. 1.

Additional experiments were performed to assess whether relevant levels of transduction are possible with highly concentrated virus. Highly concentrated stock (50 μl; concentrated 1000 fold) was added to the apical or basolateral side of ALI cultures. GFP expression was analyzed 4 days later. Consistent with observations made from the preceding set of experiments, VSV-G pseudotyped virus demonstrated transduction of, on average, 30-40% (up to 60%) of the monolayer only when applied from the basolateral side; little, if any, transduction was detected when applied apically. EboZ-pseudotyped vector demonstrated transduction of up to 40% when applied from the basolateral side and up to 70% when applied from the apical side. To control for pseudotransduction, experiments were carried out in the presence of AZT (5 μm) to inhibit RT and thus exclude GFP expression from GFP-encoding provirus. Like uninfected cultures, AZT treated cultures demonstrated no GFP expression, suggesting that pseudotransduction was not responsible for expression.

### EXAMPLE 5

### Characterization of EboZ Pseudotyped Vector

Electron microscopy (EM) was performed as follows to compare the ultrastructure of VSV-G or EboZ- to gp160 (native HIV envelope)-pseudotyped HIV vectors. The ultrastructure of membrane bound envelope proteins of immature particles was determined to minimize variation due to shedding of viral envelopes during maturation. 293T cells were triple transfected by CaPO₄ as described above. Sixty-four hours later, cells were fixed in 2.5% glutaraldehyde in phosphate-buffered saline and post-fixed in 1% osmium tetroxide. Cells were then enclosed in 1% agar, treated with 1% uranyl acetate and embedded in Epon. Ultrathin section specimens were analyzed with a Phillips transmission electron microscope at voltage of 80kV. For pre-embedding immunolabeling of Ebola virus envelope glycoprotein, transfected 293T cells were collected 72 h post-transfection, washed, and fixed with 2% paraformadehyde and 0.05% glutaraldehyde. After washing, the cells were incubated with antibody against the Ebola Zaire envelope glycoprotein, then with 15-nm gold conjugated secondary antibody. At the end of incubation, the cells were washed several times, pelleted, and refixed with 2.5% glutaraldehyde. After osmication, the cells were processed for transmission electron microscopy as above.

Using standard EM techniques, the immature gp160-pseudotyped particles exhibited a smooth viral membrane on which envelope glycoproteins were barely detectable as described previously (H. R. Gelderblom, Aids, 5:617-637 (1991)). In contrast, the EboZ and VSV-G-pseudotyped particles demonstrated an irregular envelope surface. Ebo-Z, VSV-G, and gp160 pseudotyped-particles were comparable in diameter and apparent rate of maturation. Furthermore, immunio-electron microscopy was performed with antibodies directed against the Ebola envelope glycoprotein.

These results indicate that the Ebola envelope is packaged and present at the surface of both the cells and the EboZ-HIV particles. Moreover, incorporation of EboZ does not influence the size, shape, or the maturation of the HIV vector but conferred a tropism advantage over VSV-G-pseudotyped vector for transduction of airway cells in vivo. Overall, these results suggest that EboZ-pseudotyped HIV vector can mediate gene transfer, with good efficiency, in airway epithelia of trachea and lung as well as in submucosal glands.

### EXAMPLE 6

### Transduction of Pseudotyped Vectors in Human Tracheal Explants

A. Transduction Efficiency of Pseudotyped Lentiviruses

Small pieces (0.5 cm²) were excised from explanted normal or CF human airways and placed on collagen-coated permeable supports. Tissue could be fed from the basolateral surface with media as above. Tissues were infected with 50-100 μl of highly concentrated EboZ or VSV-G-pseudotyped viruses encoding β-galactosidase (β-gal) from the apical surface and incubated for 2-4 h.

Viral titers were determined by limiting dilution on 293 T cells, demonstrating 1×10⁷ TU/ml for EboZ-LIIV and 1×10⁹ TU/ml for VSV-G-HIV vector.

Media was replaced and the tissue was then submerged in media overnight with replacement performed every 12 h. Tissue was fixed in 0.5% glutaraldehyde, stained with X-gal at 37° for 3-12 h, and processed for paraffin embedding. VSV-G-pseudotyped vector resulted in minimal expression of β-gal in the surface epithelium while EboZ-pseudotyped vector yielded staining of the epithelium when incubated in X-gal substrate 24 h post-infection). Histologic photomicrographs of these tissues stained 16 h post-infection demonstrated no specific expression in the vehicle or VSV-G pseudotyped treated tissues, and many cells positive for β-gal in the tissues transduced with EboZ pseudotyped virus. In all three tissues, there is punctate staining that is believed to be non-specific, and is distinct from the more dense staining of the perinuclear region and other cytoplasmic compartments in several epithelial cells in only the EboZ treated tissue. At 48 h after infection, diffuse cytoplasmic staining was seen in only the EboZ treated tissues, however tissue degradation was much more pronounced. Non-infected controls analyzed side-by-side yielded no X-gal staining on gross or histologic examination.

C. Transduction Efficiency of HIV-Ebo Virus

These data demonstrate that an envelope derived from the Zaire strain of the Ebola virus (EboZ), conferred strong ability to transduce human airway epithelium in vitro and ex vivo. Indeed, er vitro transduction of non-CF human trachea by EboZ-pseudotyped vector demonstrated efficient transduction as evaluated by tissue staining at 24 h post-infection. Evaluation of expression 24 h post-infection by histologic sectioning revealed high levels of β-gal expression. However, the integrity of the tissue was very poor due to the devascularized nature of the specimen. In order to confirm that EboZ-HIV was able to transduce intact epithelium, histologic sections were performed 16 h post-infection and stained for β-gal expression. These sections demonstrated specific intracellular expression as well as a healthy appearance to the epithelium. Although the expression observed in sections at 16 h was weak, probably due to early expression, many cells were transduced. These tissues demonstrate punctate staining by X-gal, some of which is non-specific, but perinuclear localization, characteristic of early β-gal expression (E. Y. Snyder, et al., Cell, 68:33-51 (1992)) can also be seen.

### EXAMPLE 7

### Intratracheal Delivery of HIV-Ebo

To further test the ability of EboZ pseudotyped virus to efficiently transduce intact airway epithelium, immunocompetent mice received intratracheal installation of vector encoding β-gal and were sacrificed at various time points (days 7.28, and 63 ) to evaluate the kinetics of expression.

C57B1/6 mice (6-8 weeks of age) were anesthetized using intraperitoneal ketamine/xylazine. Using standard techniques, the trachea was exposed through a midline incision, 100 μl of vector preparation were instilled using a syringe, and the subcutaneous tissues were sutured closed. Viral titers were determined by limiting dilution on 293T cells, demonstrating 5×10⁷-5×10⁸ TU/ml for EboZ-HIV and 5×10⁹-5×10¹⁰ TU/ml for VSV-G-HIV vector. Animals were maintained in the animal facility until necropsy. At necropsy, the lungs were inflated with OCT/PBS (1:1) and processed using cryofixation. 10 μm cryosections were prepared and stained with X-gal overnight. Transduction efficiency was estimated by examining 20-25 high powered fields from 16 cryosection is spaced throughout the tissue block (at 400 μlm intervals). Two animals were treated with each pseudotyped virus, and experiments were duplicated.

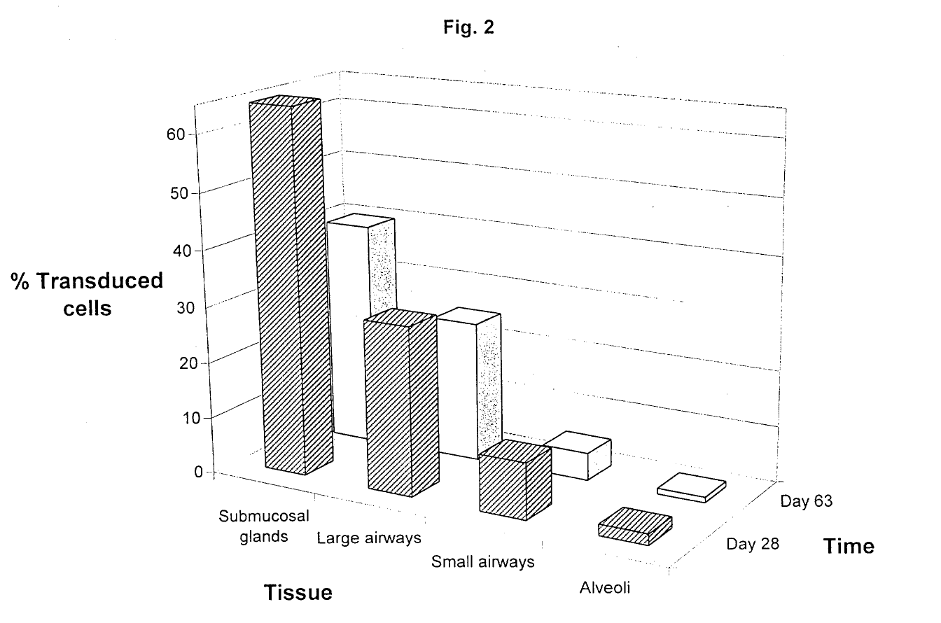

At all time points, vehicle (DMEM) treated animals demonstrated low levels of expression in the airways equivalent to background. Animals receiving VSV-G-pseudotyped vector showed similar levels of background expression in airway epithelia. In sharp contrast, animals receiving EboZ-pseudotyped vector demonstrated minimal expression at day 7, but strong expression in the airway epithelium by day 28 which persisted at day 63 (FIG. 2). Indeed, in trachea of mice that received EboZ-pseudotyped vector, regions of the airway exhibited from 0.1% to more than 80% of β-gal expressing cells after 28 days. On average, 30% of the entire tracheal epithelium was transduced by EboZ-pseudotyped HIV vector at day 28 and 24% at day 63. Interestingly, high expression was observed in submucosal glands (an average of 65% of cells) of airways from animals receiving EboZ-pseudotyped HIV virus. No submucosal gland staining was seen in control animals receiving vehicle and <1% of gland cells were positive in animals receiving VSV-G-pseudotyped vector. EboZ-pseudotyped vector mediated transduction efficiency was lower in epithelia of more distal lung (airways and alveolar cells) when compared to the trachea at day 28 (data not shown), which decreased slightly but persisted at day 63. At day 63, animals receiving EboZ-pseudotyped HIV virus demonstrated 5% of small airway cells and 1% of alveolar cells expressing β-gal.

### EXAMPLE 8

### Minimal HIV-1 Packaging Construct

An HIV-1 packaging construct was generated which contained only sequences responsible for Gag and Gag-Pol polyprotein synthesis along with 399 nucleotides of the Rev responsible element. Titers of about 10⁶ were repeatedly generated by cotransfection of this packaging construct, a Rev expressor, a VSV-G expressor and a GFP transfer vector in 293T cells. Similar results are anticipated when the HIV-1 is packaged in an Ebola capsid.

### EXAMPLE 9

### Production of Recombinant Transfer Vector Carrying CFTR Gene

Plasmid HR CFTR (HIV vector containing the CFTR gene) is prepared as follows. CFTR is isolated from AdCBCFTR by a Smal digestion. Then the Smal-Smal CFTR gene is ligated to the pHR backbone in the blunted BamHI/Xhol site by using the Klenow fragment of E coli.

Plasmid pHR′EFGP is produced as described in Example 2.

Plasmid RM (CMV/gag-pol/RRE packaging constructs, e.g., without tat, rev, vif, vpr, vpu) is generated as follows. A Xhol site is inserted by PCR mutagenesis at position 4389 in pCMVΔR8.2 to generate pCMVΔR8.2Xhel (+1 corresponds to the first nucleotide of the HIV sequence after the CMV promoter in pCMVΔR8.2). Then pCMVΔR8.2Xhel is digested with Xhol/Aval, which removed all regulatory and accessory genes as well as the envelope, and ligated to 399 nucleotides encompassing the RRE. The XhoI-RRE-AvaI fragment is generated by PCR from pCMVΔR8.2 with primers: oligo 5′ XheI (sense): SEQ ID NO: 5: 5′-AAT TGA ACC ATC TCG AGT AGC ACC C-3′ and oligo 2′ Aval (anti-sense): SEQ ID NO: 6: 5′-CCC ACT CCA TTC CGG ACT CGG GAT TCC ACC TGA-3′.

Using these constructs, a transfer virus encoding the wt CFTR gene is produced using the methods described in Example 3.

All publications cited in this specification are incorporated herein by reference. While the invention has been described with reference to a particularly preferred embodiment, it will be appreciated that modifications can be made without departing from the spirit of the invention. Such modifications are intended to fall within the scope of the appended claims.

What is claimed is:

1. A recombinant transfer virus useful for delivering a selected molecule to a host cell, said virus comprising:
a lentivirus minigene comprising lentivirus 5′ long terminal repeat (LTR) sequences, a molecule for delivery to a host cell, and a functional portion of the lentivirus 3′ LTR sequences, wherein said minigene lacks the ability to express functional lentivirus envelope proteins and is packaged in
a heterologous envelope comprising a filovirus envelope binding domain.

2. The recombinant transfer virus according to claim 1, wherein said lentivirus minigene further comprises Rev response element (RRE) sequences.

3. The recombinant transfer virus according to claim 1, wherein said lentivirus sequences are selected from the group consisting of a human immunodeficiency virus (HIV) vector, simian immunodeficiency virus (SIV) vector, caprine arthritis and encephalitis virus, equine infectious anemia virus, visna virus, and feline immunodeficiency virus (FIV) vector.

4. The recombinant transfer virus according to claim 3, wherein said lentivirus is an HIV.

5. The recombinant transfer virus according to claim 1, wherein said 5′ LTR sequences are self-inactivating.

6. The recombinant transfer virus according to claim 5, wherein said 5′ LTR sequences contain a deletion in the U3 region.

7. The recombinant transfer virus according to claim 1, wherein said 3′ LTR sequences are self-inactivating.

8. The recombinant transfer virus according to claim 7, wherein said 3′ LTR sequences contain a deletion in the U3 region.

9. The recombinant transfer virus according to claim 1, wherein said filovirus protein is an ebola envelope protein.

10. The recombinant transfer virus according to claim 1, wherein said envelope protein is a fusion protein comprising a filovirus envelope protein or fragment thereof containing the filovirus binding domain fused in frame to a second viral envelope protein or fragment thereof.

11. The recombinant transfer vector according to claim 10, wherein said fusion protein comprises a filovirus binding domain fused to the membrane domain of a second viral envelope protein.

12. The recombinant transfer vector according to claim 11, wherein said fusion protein comprises an ebola virus binding domain fused to the membrane domain of VSVG.

13. A host cell containing a recombinant transfer virus according to claim 1.

14. A method of producing a recombinant virus useful for delivering a selected molecule to a host cell, wherein said method comprises the steps of culturing in a host cell:
(a) lentiviral sequences necessary to express lentivirus gag polypeptide and lentivirus gag-pol polypeptide,
(b) a lentivirus minigene comprising lentivirus 5′ long terminal repeat (LTR) sequences, a molecule for delivery to a host cell, and a functional portion of the lentivirus 3′ LTR sequences, wherein said minigene lacks the ability to express functional lentivirus envelope proteins; and
(c) a nucleic acid molecule encoding an envelope protein comprising a filovirus binding domain under the control of regulatory sequences which direct expression of the envelope protein in the host cell,
wherein said host cell is cultured under conditions which permit packaging of the lentivirus minigene carrying the molecule in the envelope protein.

15. The method according to claim 14, wherein the host cell is a 293T cell.

16. The method according to claim 14, wherein said lentivirus minigene is carried on a plasmid.

17. The method according to claim 14, wherein the lentiviral sequences (a) are carried on a plasmid.

18. The method according to claim 8, wherein the nucleic acid molecule (c) is a plasmid.

19. A method of treating a patient with a selected molecule, said method comprising the step of transducing the cells of the patient with the recombinant virus according to claim 1.

20. The method according to claim 19, wherein the cells are selected from among the lung cells, dendritic cells and macrophages

21. The method according to claim 19, wherein said recombinant virus is administered directly to the patient.

21. The method according to claim 19, wherein the transgene is a CFTR gene and said recombinant virus is administered intratracheally.

22. The method according to claim 19, wherein the cells of the patient are transduced ex vivo, further comprising the step of re-infusing the transduced cells into the patient.

23. The method according to claim 22, wherein the patient is a cancer patient.

24. The method accordion to claim 22, wherein the transduced cells are dendritic cells.

25. The method according to claim 22, wherein the transduced cells are macrophages.

26. A method of delivering a molecule to the apical cells of the lung, said method comprising the step of administering a recombinant virus according to claim 1 intratracheally.
